(19) United States
(12) Patent Application Publication
(10) Pub. No.: US 2020/0000069 A1
(43) Pub. Date: Jan. 2, 2020

(54) PREVENTION OF DISEASES IN HONEYBEES AND REDUCTION OF PESTICIDE RESIDUES IN BEESWAX

(71) Applicant: Arkema !nc., King of Prussia, PA (US)

(72) Inventor: Christian G. Becker, King of Prussia, PA (US)

(21) Appl. No.: 16/489,397

(22) PCT Filed: Mar. 1, 2018
(86) PCT No.: PCT/US18/20432

§ 371 Date: Aug. 28, 2019

Related U.S. Application Data
(60) Provisional application No. 62/466,409, filed on Mar. 3, 2017.

(51) Int. Cl. A01K 51/00; A01N 59/00; A01N 25/02; A01N 37/16

(52) U.S. Cl. CPC A01K 51/00; A01N 37/16; A01N 25/02; A01N 59/00

(57) ABSTRACT

A beeswax and/or beehive or bee keeping equipment surface is treated with a composition containing at least one solvent, at least one surfactant and at least one oxidizing agent (e.g., peroxide), optionally also containing other components such as an oxidizing agent activator, a viscosifying agent, an enzyme and/or a pH control agent, to reduce the amount of pesticide residues on the beeswax surface and/or disinfect against any bacterial, viruses, and fungi infestation.

### FIELD OF THE INVENTION

The present invention pertains to methods for the prevention and/or treatment of diseases in honey bees, honey bee larvae and honey bee hives and/or for reducing the levels of residual pesticides in beeswax, particularly the removal of such pesticide residues from beeswax surfaces.

### BACKGROUND OF THE INVENTION

There are numerous diseases threatening honeybees and honey production. These diseases arise from sources such as bacteria, fungi, viruses, fungi protozoa and mites. In addition, contamination of bee products with pesticides used to treat pests and parasites is well known. The two principal sources of pesticide contamination mentioned in the literature are environmental (pesticides used in crop management) and apicultural practices (treatment of pests in beehives) [Contaminants of bee products, S. Bogdanov, Apidologie 37 (2006), 1-18].

According to the United States Department of Agriculture, Animal and Plant Health Inspection Service, Survey of Honey Bee Pests and Diseases, Apr. 7, 2017, honey bee health decline has been documented for years. In recent years, winter losses have been unsustainably high ranging from 22% to 36% nationally. These rates of loss threaten the viability of beekeeping operations and the production of crops dependent on bees for pollination as well as honey production.

In addition to pests and parasites, honeybees are also subject to a multitude of diseases:

bacterial diseases such as American foulbrood, European foulbrood, fungal diseases such as Chalkbrood; Stonebrood; Nosema, and viral diseases such as Cripaviridae—Chronic bee paralysis virus, Dicistroviridae—Acute bee paralysis virus, Israeli acute paralysis virus, Kashmir bee virus, Black queen cell virus; Cloudy wing virus; Sacbrood virus; Iflaviridae—Deformed wing virus, Kakugo virus; Iridoviridae—Invertebrate iridescent virus type 6, Secoviridae—Tobacco ringspot virus; Lake Sinai virus.

American foulbrood (AFB) is one of the most virulent brood diseases known in honeybees. The disease is caused by the spore forming bacterium Paenibacillus larvae. American foulbrood spores are extremely resistant to desiccation and can remain viable for more than 40 years in honey and beekeeping equipment. Each dead larva may contain as many as 100 million spores. Because the spores can remain viable for years, many countries require bee colonies with AFB to be burned. Other countries (e.g., USA, Canada, and Argentina) allow the use of antibiotics to keep the disease in control.

Nosemosis is by far the most widespread and the most damaging adult bee disease. Infections are acquired by the uptake of spores during feeding or grooming. Nosema apis is a microsporidian, a small, unicellular parasite recently reclassified as a fungus that mainly affects honey bees. It causes nosemosis, also called nosema, which is the most common and widespread of adult honey bee diseases. Actually, nosema is so wide-spread that it is presumed that every colony has some infected bees. It is often treated with antibiotics.

Treating with antibiotics in order to prevent or save colonies from diseases is obviously an issue as it introduces long lasting chemical residues in the beehive, which in turns can end up in honey and wax frames. In the EU, honeybees are classified as food producing animals. Accordingly, a maximum residue limit (MRL) for honey must be met before a marketing authorization can be granted. So, in principle, only medicinal products which do not result in residues in honey could be authorized.

This is obviously a problem for trade as some countries are banning the use of antibiotics and some countries authorize antibiotics only under specific circumstances. It is also very troubling that antibiotics could be present in beeswax and honey.

Non-limiting examples of antibiotics or chemotherapeutics used in various countries are Streptomycin, Tetracyclines, Sulfonamides, Erythromycin, Tylosin, Lincomycin, Enrofloxacin, Ciprofloxacin, Trimethoprim, Metronidazole, Choramphenicol, Nitrofurans. [Antimicrobials in beekeeping—W. Reybroeck, E. Daeseleire, H. De Brabander, L. Herman, Veterinary Microbiology 158 (2012) 1-11].

Risks related to the use of antibiotics for the control of honeybee diseases are persistence of the infection, reappearance of the disease and honey contamination. It would therefore be advantageous to be able to treat against bacterial, viruses, fungi, protozoa and mites using a product that would not leave any chemical residues. The present invention is directed towards the use of oxidizing agents such as hydrogen peroxide to provide both the sanitation effect and zero residue aspect of the treatment. Treatment can be done by spraying, immersion or fogging of the equipment.

Disinfection of beehives and beehive equipment using non-antibiotic products was proposed and disclosed in prior art. United States Patent Publication No. 2009/0104288 discloses the use of a hop derivative to treat beehives. U.S. Pat. No. 6,096,350 discloses the treatment of honey bee by applying an effective amount of an aqueous composition comprising a protic acid and a chlorite ion. United States Patent Publication No. 2009/01182143 discloses increasing the tolerance of bees to disease by feeding bees an effective amount of the nucleic acid agent comprising a nucleic acid sequence down regulating expression of a gene product of a bee pathogen. While all these methods have merit, they are either complicated, expensive or difficult to implement. In addition, residues of the solution added to the beehives are expected to be present after treatment.

Contaminants can reach the raw materials of bee products when transported into the beehives by the bees after foraging pollen, nectar and water from plants that have been sprayed with pesticides (or added to the vegetation by other means such as seed treatments) by farmers, agrochemical professionals, gardeners and the likes in order to control a variety of agricultural pests that can damage crops.

However, the most important contaminants are probably the substances used in the control of honey bee pests. There are many bee pests, parasites and diseases (Varroa mites, tracheal mites, small hive beetles, wax moths, tropilaelaps, nosema disease, American and European foulbrood, and so forth).

At present, one of the most important pests worldwide is Varroa destructor. Varroa destructor (varroa mite) is an external parasitic mite that attacks the honey bees Apis cerana and Apis mellifera. The disease caused by the mites is called varroosis. Acaricides used in the control of Varroa destructor are a major source of pollution because they often involved slow release products and must be present in the beehives for a period up to 45 days to be effective and prevent re-infestation [The concentration effect of selected acaricides present in beeswax foundation on the survival of Apis mellifera colonies, S. Medici, A. Castro, E. Sralo, J. Marioli, M. Eguaras, Journal of Apicultural Research 51(2):164-168 April 2012].

Both the unintentional and the intentional exposures of honey bees (and other pollinators) to pesticides have resulted in pesticide residues being detected in the beehives, bees, pollen, honey and especially beeswax (brood nest wax and beeswax foundation). The roles of these pesticides and their residues in hive products may have played a role in colony collapse disorder (CCD) and in other colony problems observed in the last several years [Pesticides and honey bee toxicity—USA, R. M. Johnson, M. D. Ellis, C. A. Mullin, M. Frazier, Apidologie, 41, issue 3, (2010), 312-331]. Colony collapse disorder causes significant economic losses because many agricultural crops worldwide are pollinated by western honey bees. Although the causes for bee declines are diverse and not always well understood, honey bee exposure to pesticides can have a severe impact.

Chronic exposure to pesticides have long been suspected as a potential cause of honey bee declines. In a recently published study [High Levels of Miticides and Agrochemicals in North America Apiaries: Implications for Honey Bee Health, C. Mullin, M. Frazier, J. Frazier, S. Ashcraft, R. Simonds, D. vanEngelsdorp, J. Pettis, PLoS ONE, march 2010, vol. 5, issue 3, e9754], most comb and foundation waxes sampled were found contaminated with 87 diverse pesticides and metabolites, with up to 39 different detections in a single sample, averaging 8 different pesticide residues each. The most frequent detections were the in-hive acaricides fluvalinate and coumaphos (two very common miticides), the organophosphate pesticide chlorpyriphos and chlorothalonil, a widely used fungicide.

Examples of typical miticide commercial products used in beehives are fluvalinate (Apistan® anti-varroa mite strips), amitraz (Apivar®), or coumaphos (Checkmite+® beehive pest control strip). A complete list of such chemicals can be found on the Environmental Protection Agency (EPA) registered pesticide products list approved specifically for use in beehives and on the list of pesticides approved for application on the various life stages of crops.

As used herein, the term “pesticide” includes all of the following: herbicides, insecticides, insect growth regulators, nematicides, termiticides, molluscicides, piscicides, avicides, rodenticides, predacides, bactericides, insect repellents, animal repellents, antimicrobials, fungicides, disinfectants (antimicrobials), and sanitizers.

Most pesticides, fungicides and acarides are lipophilic or fat soluble, non-volatile and persistent, and thus easily accumulate in the beeswax. It is common beekeeping practice to recycle beeswax almost continuously. As a consequence, pesticides can be accumulated over a period of several years creating an unhealthy toxic environment for the bees and brood. Additionally, pesticides often resist degradation at the wax melting temperatures used during wax processing. It has been shown that a purification process involving melting the beeswax in boiling water does not substantially modify the initial content of the lipophilic contaminants in beeswax when these are present in relatively high concentrations [Residues of Organic contaminants in beeswax, J. J. Jimenez, J. L. Bernal, M. J. del Nozal, M. T. Martin, Eur. J. Lipid Sci. Technol. 107 (2005) 896-902].

Having wax foundations made of clean, pesticide residue-free wax would be greatly beneficial to the apicultural world. The current literature lists a few attempts by beekeepers and researchers to find a solution to this issue. In a similar approach, formulations with peroxide compounds have been proposed for the decontamination of warfare agents [Universal decontaminating solution for chemical warfare agents, G. Wagner and Y. Yang, U.S. Pat. No. 6,245,957]. One publication also indicated the mineralization of pesticides using the photo-Fenton reaction [Degradation of selected pesticide active ingredients and commercial formulations in water by the photo-assisted Fenton reaction, P. Huston and J. Pignatello, Wat. Res. Vol. 33, No. 5, pp. 1238-1246, 1999]. Ozone has also been mentioned as decreasing pesticide residues in honey bee combs [The potential for using ozone to decrease pesticide residues in honey bee comb, R. James, J. Ellis and A. Duehl, Agricultural Science, Vol. 1 Issue 1 (2013), pp 1-16]. The reduction of fluvalinate residues in beeswax by chemical means, for example, was found unsuccessful when the beeswax is bleached with hydrogen peroxide as the peroxide reacted with the more reactive unsaturated fatty acids instead of the fluvalinate [A Review of Treatment Options for Control of Varroa Mite in New Zealand, Report to the Ministry of Agriculture and Forestry (MAF), by The Horticulture & Food Research Institute of New Zealand Ltd, HortResearch Client Report No. 2001/249; Reduction of Fluvalinate Residues in Beeswax by Chemical Means, V. Veslý, M. Máchová, J. Hessler, V. Hostomská & J. Leníc̆ek, Journal of Apicultural Research, 33:3, 185-187, (1994)]. None of these treatments particularly target the wax surface which is in direct contact with the bees. Hence, there apparently are no successful treatment methods known in the art for the reduction of pesticide residues present on a beeswax surface and just beneath the surface (sub-surface) using a formulation based on peroxide.

### SUMMARY OF THE INVENTION

Compositions containing one or more surfactants, one or more solvents, and one or more oxidation (oxidizing) agents such as peroxide compounds, with or without activators, may be used to treat against bacterial, viruses, fungi, protozoa and mite infestation and/or to reduce the levels of pesticide residues on the surface of beeswax surfaces. The pesticide residues may, in certain embodiments, be converted into less harmful substances. A reduction in pesticide residue levels allows the honeybees to build new structure on cleaner wax. The composition can, for example, be sprayed onto the wax surface to be decontaminated, or equipment containing the wax can be submerged in the composition. In another application, the composition can be fogged or vaporized on the equipment or inside a beehive. Passive evaporation as well as other means known in the art of applying a product, particularly a liquid product, on a surface are also contemplated herein.

Oxidizing agents such as hydrogen peroxide is not only well known against pathogens in the medical environment but also benefits from a complete decomposition of the product into oxygen and water. It is noteworthy that hydrogen peroxide is already naturally present in small quantity in honey. It was reported that the antibacterial activity of honey is attributable to hydrogen peroxide produced by the bee-derived enzyme glucose oxidase. Thus, the issue that the treatment with hydrogen peroxide could in theory leave traces of hydrogen peroxide in the honey is not believed to be problematic.Various aspects and embodiments of the invention may be summarized as follows:

Aspect 1: A method of reducing pesticide residues in beeswax, comprising, consisting essentially of or consisting of contacting a beeswax surface contaminated with said pesticide residues with a composition comprised of, consisting essentially of or consisting of at least one solvent, at least one oxidizing agent and at least one surfactant.

Aspect 2: The method of Aspect 1, wherein the composition is comprised of at least one peroxide as an oxidizing agent.

Aspect 3: The method of Aspect 2, wherein the composition is additionally comprised of at least one peroxide activator.

Aspect 4: The method of Aspect 3, wherein the at least one peroxide activator is applied separately to the beeswax surface in a different step than the at least one peroxide.

Aspect 5: The method of Aspect 3 or 4, wherein the at least one peroxide activator comprises, consists essentially of or consists of at least one peroxide activator selected from the group consisting of metal-containing peroxide activators, carbonate salts and combinations thereof.

Aspect 6: The method of any of Aspects 3-5, wherein the composition is comprised of from about 0.001% to about 20% by weight or from about 0.001% to about 5% by weight peroxide activator.

Aspect 7: The method of any of Aspects 1-6, wherein the composition is additionally comprised of at least one enzyme capable of degrading at least a portion of the pesticide residues.

Aspect 8: The method of Aspect 7, wherein the composition is comprised of from about 0.1% to about 20% or from about 0.1 to about 5% by weight enzyme.

Aspect 9: The method of any of Aspects 1-8, wherein the at least one solvent comprises, consists essentially of or consists of at least one solvent selected from the group consisting of water, water-miscible or partially water-miscible organic solvents and combinations thereof.

Aspect 10: The method of any of Aspects 1-9, wherein the at least one solvent comprises, consists essentially of or consists of at least one water-miscible or partially water-miscible organic solvent selected from the group consisting of alcohols, ethers, esters and ketones.

Aspect 11: The method of any of Aspects 1-10, wherein the at least one solvent comprises, consists essentially of or consists of at least one water-miscible or partially water-miscible organic solvent selected from the group consisting of carbonate esters.

Aspect 12: The method of any of Aspects 1-11, wherein the composition is comprised of from about 0.1% to about 90% or from about 1% to about 60% by weight solvent.

Aspect 13: The method of any of Aspects 1-12, wherein the composition comprises, consists essentially of or consists of at least one peroxide selected from the group consisting of hydrogen peroxide, peroxyacids, peroxycarbonates, urea hydrogen peroxide, perborate compounds, and combinations thereof.

Aspect 14: The method of any of Aspects 1-13, wherein the composition is comprised of from about 0.1% to about 70%, from about 1% to about 15%, or from 1% to about 8% by weight peroxide.

Aspect 15: The method of any of Aspects 1-14, wherein the at least one surfactant comprises, consists essentially of or consists of at least one surfactant selected from the group consisting of anionic surfactants, cationic surfactants, nonionic surfactants, amphoteric surfactants and combinations thereof.

Aspect 16: The method of any of Aspects 1-15, wherein the at least one surfactant comprises, consists essentially of or consists of at least one surfactant selected from the group consisting of organomodified siloxane non-ionic surfactants, polyalkoxylated sorbitan carboxylates, alkyl sulfate surfactants, alcohol ethoxylate surfactants, polysorbate surfactants and combinations thereof.

Aspect 17: The method of any of Aspects 1-16, wherein the composition is comprised of from about 0.01% to about 30% or from about 0.01% to about 10% by weight surfactant.

Aspect 18: The method of any of Aspects 1-17, wherein the contacting is carried out at a temperature of from about 10° C. to about 50° C.

Aspect 19: The method of any of Aspects 1-18, wherein the contacting is carried out for a time of from about 1 minute to about 1 hour or until such time as a layer of the composition deposited on the beeswax surface dries up.

Aspect 20: The method of any of Aspects 1-19, wherein the contacting is achieved by spraying the composition onto the beeswax surface, immersing the beeswax surface in the composition, or fogging the beeswax surface with the composition.

Aspect 21: The method of any of Aspects 1-20, wherein the composition is activated by subjecting the composition to cold plasma ionizing during a fogging process before the composition reaches the beeswax surface (wherein the cold plasma ionization results in the generation of free radicals in the composition, particularly where the oxidizing agent is a peroxide).

Aspect 22: The method of any of Aspects 1-21, wherein the pesticide residues are comprised of one or more pesticides selected from the group consisting of herbicides, insecticides, insect growth regulators, nematicides, termiticides, molluscicides, piscicides, avicides, rodenticides, predacides, bactericides, insect repellents, animal repellents, antimicrobials, fungicides, disinfectants (antimicrobials), and sanitizers.

Aspect 23: The method of any of Aspects 1-22, wherein the pesticide residues are comprised of one or more pesticides selected from the group consisting of carbamates, organophosphates, pyrethroids, neonicotinoids, strobilurin, fluvalinate, amitraz, coumaphos, chlorothalanil, chlorpyriphos, endosulfan, pendimethalin, fenpropathrin, esfenvalerate, azoxystrobin, methoxyfenozide, atrazine, bifenthrin, dicofol, aldicarb sulfoxide, trifluralin, boscalid, carbendazim, and combinations thereof.

Aspect 24: The method of any of Aspects 1-24, wherein the composition is additionally comprised of at least one viscosifying agent and/or gelling agent.

Aspect 25: The method of any of Aspects 1-24, wherein the composition is comprised of from about 0.01% to about 10.0% by weight or from about 0.1% to about 5.0% by weight in total of viscosifying agent and/or gelling agent.

Aspect 26: A method for preventing or reducing pathogens in beehives and beehive equipment, comprising contacting beehive or beehive equipment with a composition comprised of at least one solvent, at least one oxidizing agent and at least one surfactant.

Aspect 27: The method of aspect 26, wherein the composition is comprised of at least one peroxide as an oxidizing agent.

Aspect 28: The method of aspect 27, wherein the composition is additionally comprised of at least one peroxide activator.

Aspect 29: The method of aspect 27, wherein the at least one peroxide activator comprises, consists essentially of or consists of at least one peroxide activator selected from the group consisting of metal-containing peroxide activators, carbonate salts and combinations thereof.

Aspect 30: The method of aspect 28 or 29, wherein the composition is comprised of from about 0.001% to about 20% by weight or from about 0.001% to about 5% by weight peroxide activator.

Aspect 31: The method of aspect 26 - 30, wherein the at least one solvent comprises, consists essentially of or consists of at least one solvent selected from the group consisting of water, water-miscible or partially water-miscible organic solvents and combinations thereof.

Aspect 32: The method of aspect 26-31, wherein the at least one solvent comprises, consists essentially of or consists of at least one water-miscible or partially water-miscible organic solvent selected from the group consisting of alcohols, ethers, esters and ketones.

Aspect 33: The method of aspect 26-32, wherein the at least one solvent comprises, consists essentially of or consists of at least one water-miscible or partially water-miscible organic solvent selected from the group consisting of carbonate esters.

Aspect 34. The method of aspect 26-33, wherein the composition is comprised of from about 0.1% to about 90% or from about 1% to about 60% by weight solvent.

Aspect 35: The method of aspect 27-33, wherein the at least one peroxide comprises, consists essentially of or consists of at least one peroxide selected from the group consisting of hydrogen peroxide, peroxyacids, peroxycarbonates, urea hydrogen peroxide, perborate compounds, and combinations thereof.

Aspect 36: The method of aspect 27-35, wherein the composition is comprised of from about 0.1% to about 70%, from about 1% to about 15%, or from 1% to about 8% by weight peroxide.

Aspect 37: The method of aspect 26-36, wherein the at least one surfactant comprises, consists essentially of or consists of at least one surfactant selected from the group consisting of anionic surfactants, cationic surfactants, nonionic surfactants, amphoteric surfactants and combinations thereof.

Aspect 38: The method of aspect 26-37, wherein the at least one surfactant comprises, consists essentially of or consists of at least one surfactant selected from the group consisting of organomodified siloxane non-ionic surfactants, polyalkoxylated sorbitan carboxylates, alkyl sulfate surfactants, alcohol ethoxylate surfactants, polysorbate surfactants and combinations thereof.

Aspect 39: The method of aspect 26-38, wherein the composition is comprised of from about 0.01% to about 30% or from about 0.01% to about 10% by weight surfactant.

Aspect 40: The method of aspect 26-39, wherein the contacting is achieved by spraying the composition onto the beehive or beehive equipment, immersing the beehive or beehive equipment in the composition, or fogging the beehive or beehive equipment with the composition.

Aspect 41: The method of aspect 26-40 wherein the composition is activated by subjecting the composition to cold plasma ionizing during a fogging process before the composition reaches the beehive or beehive equipment.

Aspect 42: The method of aspect 26-41, wherein the composition further comprises at least one viscosifying agent and/or gelling agent.

Aspect 43: The method of aspect 26-42, wherein the composition is comprised of from about 0.01% to about 10.0% by weight or from about 0.1% to about 5.0% by weight in total of viscosifying agent and/or gelling agent.

Aspect 44: The method of claim aspect 26-43 where the pathogens are responsible for a disease selected from the group consisting of the American Foulbrood (AFB) disease and Nosema disease.

Aspect 45: The method of aspect 26-44 wherein the pathogens are responsible for: bacterial diseases selected from the group consisting of American foulbrood, and European foulbrood; fungal diseases selected from the group consisting of Chalkbrood, Stonebrood, and Nosema; and viral diseases selected from the group consisting of Cripaviridae, Chronic bee paralysis virus, Dicistroviridae, Acute bee paralysis virus, Israeli acute paralysis virus, Kashmir bee virus, Black queen cell virus, Cloudy wing virus, Sacbrood virus; Iflaviridae—Deformed wing virus, Kakugo virus; Iridoviridae—Invertebrate iridescent virus type 6, Secoviridae—Tobacco ringspot virus, and Lake Sinai virus.

Aspect 46: The method of aspect 26-45 wherein the pathogens are bacteria selected from the group consisting of Melissococcus plutonius, Paenibacillus larvae, Spiroplasma apis, S. melliferum, Pseudomonas aeruginosa, Achromobacter euridice, Enterococcus faecalis, Paenibacillus alvei, and Brevibacillus laterosporus.

Aspect 47: The method of aspect 26-46 wherein the pathogens are fungi selected from the group consisting of Nosema apis, Nosema ceranae, Ascosphaera apis, and Aspergillus spp.

Aspect 48: The method of aspect 26-47 wherein the pathogens are viruses selected from the group consisting of Israeli acute paralysis virus, acute bee paralysis virus, Kashmir bee virus, black queen cell virus, deformed wing virus/Kakugo virus, Varroa destructor virus, sacbrood virus slow bee paralysis virus, chronic bee paralysis virus and Lake Sinai virus.

Aspect 49: The method of aspect 26-48 wherein the pathogens are the bacteria spores or fungi spores.

### DETAILED DESCRIPTION OF CERTAIN EMBODIMENTS OF THE INVENTION

### Surfactants

The composition utilized in the process of the present invention is comprised of at least one surfactant (surface active agent). Two or more surfactants may be employed in certain embodiments of the invention. The surfactant or surfactants may be selected to be effective in wetting a beeswax,beehive or beekeeping equipment surface and allowing good penetration of the composition on the entire surface to be treated, thereby helping to lift pesticide residues (and/or oxidation or decomposition products thereof) off the surface or disinfect the surface. For example, the surfactant(s) may assist in solubilizing and/or emulsifying the pesticide residues and/or decomposition products thereof formed by reaction of the pesticide residues with oxidizing agent (e.g., peroxide). Any known type of surfactant or combination of known surfactant types may be employed such as, for example, anionic surfactants, cationic surfactants, nonionic surfactants, amphoteric surfactants and combinations thereof. Suitable surfactants, only provided herein as non-limiting examples, include organomodified siloxanes (e.g., the surfactants sold by Evonik under the brand name Break-thru® or the surfactant sold by Dow Corning under the brand name Xiameter® OFX-0309 fluid), alcohol ethoxylate surfactants (e.g., the surfactants sold by Air Products under the brand name Tomadol®), polysorbate surfactants (e.g., the surfactant sold by Croda under the brand name Tween™ 20) and alkyl sulfates such as ammonium lauryl sulfate (ALS). A variety of surfactants (which may also be referred to as wetting agents) as well as combinations of surfactants (wetting agents) can be selected, keeping in mind that such surfactants should advantageously be stable and compatible with the other components of the composition, nontoxic to honey bees and other pollinators, and environmentally friendly at the selected use rate. In various embodiments of the invention, the surfactant(s) is or are present in the composition at a concentration of 0.01 to 30% by weight or 0.01 to 10% by weight.

### Solvents

One or more various solvents that are compatible with the other components of the composition can be used. Water, but also organic solvents or a combination or water and organic solvents in various proportions, can be present in the composition to be employed in the pesticide reduction and/or disinfection method of the present invention. Suitable illustrative organic solvents include, but are be limited to, alcohols in particular C2-C6 aliphatic mono-alcohols and glycols such as ethanol, isopropanol, tert-butanol, propylene glycol and derivatives thereof (e.g., glycol ethers). Other suitable organic solvents include organic carbonates, particularly propylene carbonate. The role of the solvent in the composition may be both to act as a carrier for the other components of the composition and to aid in solubilizing lipophilic pesticide residues present on the surface to be decontaminated and/or the derivative byproducts formed by reaction of the oxidizing agent(s) with the pesticide residues. The total amount of solvent in the composition may be, for example, between 0.1% and 90% or between 1% and 60% by weight, in various exemplary embodiments of the invention. The solvent or combination of solvents present in the composition, and the amount(s) thereof, may be selected, in coordination with the other components, so as to provide the composition in the form of a solution or an emulsion (e.g., a water-in-oil emulsion or an oil-in-water emulsion).

### Oxidizing Agents

The composition used to reduce pesticide residues and/or disinfect the beehives/bee keeping equipment in accordance with the present invention includes at least one oxidizing agent, which may be any organic or inorganic compound capable of oxidizing one or more of the compounds present in the pesticide residues present on the beeswax surface or disinfect, that is treat against any bacterial, viruses, fungi, protozoa and mite infestation. Oxidizing agents can be selected from a variety of peroxides, for example. such as, but not limited to, inorganic and organic peroxides such as hydrogen peroxide or peroxide generating compounds such as percarboxylic acids (e.g., peracetic acid), peroxycarbonates, urea hydrogen peroxide, perborate compounds, as well as similar compounds and/or a combination of such compounds. In certain embodiments of the invention, the oxidizing agent or combination of oxidizing agents is soluble in the solvent(s) present in the composition. The concentration of oxidizing agent in the composition may be between 0.1 and 70%, or between 1 and 15%, or between 1 and 8% by weight.

### Activators

The composition can be activated by the presence of an activator for the oxidizing agent, i.e., a substance that assists in catalyzing or otherwise promoting oxidation of pesticide residues or in disinfecting against any bacterial, viruses, fungi, protozoa and mite infestation. For example, the activator may convert the oxidizing agent into a more reactive substance, e.g., a substance better able to oxidize the pesticide residues than the oxidizing agent itself or better able to disinfect against any bacterial, viruses, fungi, protozoa and mite infestation. In certain embodiments of the invention, the oxidizing agent formulated into the composition may be regarded as an oxidizing agent precursor, which by itself has little or no reactivity towards the pesticide residues or disinfection impact but which is transformed in situ to a reactive oxidizing agent through interaction with an activator as described herein. Post-addition of an activator to a composition in accordance with the present invention may be practiced. The activator may be added to the composition right before application of the composition to a beeswax surface or independently applied to the surface to be treated (e.g., prior to, simultaneous with or following the application of the composition to the surface of the beeswax). Activators can be, for example, metal-containing substances such iron oxide (Fenton reaction), carbonate compounds (e.g., salts of carbonic acid such as potassium or sodium bicarbonate), a more complex activator such as Fe-TAML (tetra-amido macrocyclic ligand) developed at Carnegie Mellon or any activator or combination of activators that are known to induce the formation of free radical compounds under various conditions (including the use of UV light). The concentration of activator in the composition may be, for example, between 0.001% and 20% by weight or between 0.001% and 5% by weight.

In one embodiment of the invention, the incorporation of an activator in the composition is avoided and the composition instead activated by subjecting the composition to a physical treatment step such as exposing the composition to a source of energy such as electric or photonic energy (e.g., cold plasma ionization). For example, where the oxidizing agent is a peroxide, cold plasma ionization may generate free radical species or other highly reactive species from the peroxide, wherein the free radical species or other highly reactive species are more reactive towards the pesticide residues than the starting peroxide. In one embodiment, the composition is applied to a beeswax surface by fogging, wherein the vaporized composition is subjected to cold plasma ionization.

### pH Control Agents

The pH of the composition may be adapted or adjusted to fall within a desired or advantageous pH range (e.g., a pH of about 2 to about 10) with the addition of any acid and/or base as a pH control agent, to improve or optimize the effectiveness or performance of the composition in reducing levels of pesticide residues present in a beeswax surface and/or disinfecting against any bacterial, viruses, fungi, protozoa and mite infestation. The amount and type of acid and/or base are not particularly limited. For example, weak and/or strong acids, organic and/or inorganic acids, weak and/or strong bases, and/or organic and/or inorganic bases may be employed. The composition may be formulated to include a buffer system as a pH control agent.

### Enzymes

The compositions used in the pesticide reduction method of the present invention may also contain one or more enzymes, which may be selected to be broad acting or specific to particular targeted pesticides or pesticide families and/or bacterial, viruses, fungi, protozoa and mites. Such enzymes may be part of the composition as initially brought into contact with a beeswax surface or added after application. In various embodiments of the invention, the enzyme concentration may be between 0.1 and 20% by weight or between 0.1 and 5% by weight.

### Viscosifying Agents/Gelling Agents

The composition can be gelled or otherwise increased in viscosity for better retention on the beeswax,beehive and/or bee keeping equipment surface using at least one viscosifying agent, which may be an inorganic viscosifying agent or an organic viscosifying agent or a combination of viscosifying agents. Examples of suitable viscosifying agents include, for example, modified silicas (e.g., the silicas sold by Evonik under the brand names Aerosil® and Sipernat®), high molecular weight crosslinked polyacrylic acid polymers (e.g., the polymers sold by Lubrizol under the brand name Carbopol®), xanthan gums (e.g., the xanthan gums sold by CP Kelco under the brand name Kelzan®) and other such gums (guar gums, alginates and the like), polyol and polyether glycol compounds such as glycerol, polyethylene glycol, polypropylene glycol, and all other viscosifying or gelling agents that are compatible with the other components of the composition and preferably nontoxic to bees and environmentally friendly (e.g., non-persistent and/or biodegradable). The viscosifying agent and/or gelling agent may, in certain embodiments of the invention, act as a thixotropic agent. In various embodiments of the invention, the viscosifying or gelling agent concentration in the composition is between 0.01% and 20% by weight or between 0.1% and 5% by weight.

### Formulation of the Composition

The order and manner in which the above-described components of the composition are combined and formulated are not believed to be particularly limited. Methods and techniques known in the art may be adapted and modified as appropriate, depending upon the types and relative amounts of the components selected for use. In certain embodiments, the composition may be a one-part formulation, having sufficient physical and chemical stability to permit storage at normal conditions (e.g., in drums, tanks or other containers at room temperature) for extended periods of time. Such formulations may then be directly utilized to reduce pesticide residues on beeswax and/or beehive or bee keeping equipment surfaces in accordance with any of the procedures described herein, whereby the composition is contacted with a beeswax surface and/or beehive or bee keeping equipment for a desired amount of time. In other embodiments, the composition may be provided as a multi-part formulation, particularly where certain components of the composition are reactive with each other and it is desired to avoid such reaction until such time as the composition is to be contacted with the beeswax surface and/or beehive or bee keeping equipment. For example, where the composition comprises both a peroxide and a peroxide activator, the peroxide activator may undesirably react prematurely with the peroxide or otherwise transform the peroxide such that the effectiveness of the composition in reducing pesticide residues on beeswax and/or beehive or bee keeping equipment surfaces is reduced if the composition is stored for a long period of time prior to use. In such cases, the composition may comprise two parts which are stored separately, wherein one part comprises the peroxide (and optionally one or more components of the composition other than the peroxide activator) and a second part comprises the peroxide activator (and optionally one or more components of the composition other than the peroxide). The two parts are then combined, in the desired proportions, to obtain the composition, either shortly before application of the composition to a beeswax surface or simultaneous with contacting the beeswax surface. In yet another embodiment, a first part of the composition may be first brought into contact with the beeswax surface, followed by the second part of the composition (which may admix with the first part of the composition in contact with the beeswax surface).

### Methods of Use

In accordance with the present invention, a composition corresponding to the above description is brought into contact with a beeswax surface and/or beehive or bee keeping equipment. The contacting is carried out for a time effective to cause a measurable reduction in the level(s) of one or more pesticides present on or in the surface of the beeswax and/or any bacterial, viruses, and fungi infestation. For example, the concentration(s) of one or more pesticides at the beeswax and/or beehive or bee keeping equipment surface may be reduced at least 10%, at least 20%, at least 30%, at least 40%, at least 50%, at least 60%, at least 70%, at least 80%, at least 90% or even 100%. Typically, contact times of from about 1 minute to about 1 hour may be employed, but shorter or longer times may also be practiced. The composition may be heated to at least some extent to accelerate the rate at which pesticide residues at the surface of the beeswax are reduced. However, temperatures at which the beeswax would melt generally should be avoided (beeswax has a melting point of about 62-64° C.). For example, the contacting may be carried out at a temperature of between about 10° C. to about 50° C.

Generally speaking, the compositions employed in the method of the present invention are liquid in form and therefore any of the techniques known in the art for contacting a liquid composition with a surface may be utilized herein. For example, the composition may be contacted with a beeswax surface and/or beehive or bee keeping equipment by spraying (including spraying at high pressures), immersion (e.g., dipping), fogging (vaporizing), washing, pouring, wiping, spreading, brushing, and the like. The composition may be agitated while in contact with the beeswax and/or beehive or bee keeping equipment surface, including stirring the composition, scrubbing or rubbing the beeswax and/or beehive or bee keeping equipment surface with the composition (using a brush, sponge, abrasive pad or other scrubbing medium, for example). The composition may be formulated with an abrasive substance, so as to promote cleaning of the beeswax and/or beehive or bee keeping equipment surface. The beeswax and/or beehive or bee keeping equipment surface may be successively contacted with multiple portions of the composition (wherein the composition portions may be the same as or different from each other).

Following the contacting step, one or more further steps may be performed. For example, the composition may be drained, wiped or otherwise removed from the beeswax and/or beehive or bee keeping equipment surface. The beeswax and/or beehive or bee keeping equipment surface may be dried (e.g., air-dried or dried by warm air); any residual composition may thus be dried in place on the beeswax and/or beehive or bee keeping equipment surface. The beeswax surface may be rinsed with a suitable solvent, such as water, an organic solvent or mixture of organic solvents, or a mixture of water and one or more organic solvents (the organic solvent(s) may be the same as or different from the organic solvent(s) present in the composition; preferably, the solvent is selected to be one that does not dissolve beeswax from the beeswax and/or beehive or bee keeping equipment surface under the rinsing conditions). Following a rinse step, the beeswax and/or beehive or bee keeping equipment surface may be dried.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the composition or the method for using the composition. Additionally, in some embodiments, the invention can be construed as excluding any element or process step not specified herein.

Although the invention is illustrated and described herein with reference to specific embodiments, the invention is not intended to be limited to the details shown. Rather, various modifications may be made in the details within the scope and range of equivalents of the claims and without departing from the invention.

1. A method of treating beeswax, beehives and/or beehive equipment to reduce pesticide residue and/or prevent or reduce pathogens comprising contacting a contaminated beeswax, beehive and/or beehive equipment surface with a composition comprised of at least one solvent, at least one peroxide and at least one surfactant.

2. (canceled)

3. The method of claim 1, wherein the composition is additionally comprised of at least one peroxide activator.

4. The method of claim 3, wherein the at least one peroxide activator is applied separately to the beeswax, beehive and/or beehive equipment surface in a different step than the at least one peroxide.

5. The method of claim 3, wherein the at least one peroxide activator comprises at least one peroxide activator selected from the group consisting of metal-containing peroxide activators, carbonate salts and combinations thereof.

6. The method of claim 3, wherein the composition is comprised of from about 0.001% to about 20% by weight or from about 0.001% to about 5% by weight peroxide activator.

7. The method of claim 1, wherein the composition is additionally comprised of at least one enzyme capable of degrading at least a portion of the pesticide residues.

8. The method of claim 7, wherein the composition is comprised of from about 0.1% to about 20% or from about 0.1 to about 5% by weight enzyme.

9. The method of claim 1, wherein the at least one solvent comprises at least one solvent selected from the group consisting of water, water-miscible or partially water-miscible organic solvents and combinations thereof.

10. The method of claim 1, wherein the at least one solvent comprises at least one water-miscible or partially water-miscible organic solvent selected from the group consisting of alcohols, ethers, esters and ketones.

11. The method of claim 1, wherein the at least one solvent comprises at least one water-miscible or partially water-miscible organic solvent selected from the group consisting of carbonate esters.

12. The method of claim 1, wherein the composition is comprised of from about 0.1 % to about 90% or from about 1% to about 60% by weight solvent.

13. The method of claim 1, wherein the at least one peroxide comprises at least one peroxide selected from the group consisting of hydrogen peroxide, peroxyacids, peroxycarbonaies, urea hydrogen peroxide, perborate compounds, and combinations thereof.

14. The method of claim 1, wherein the composition is comprised of from about 0.1 % to about 70%, from about 1% to about 15%, or from 1% to about 8% by weight peroxide.

15. The method of claim 1, wherein the at least one surfactant comprises at least one surfactant selected from the group consisting of anionic surfactants, cationic surfactants, nonionic surfactants, amphoteric surfactants and combinations thereof.

16. The method of claim 1, wherein the at least one surfactant comprises at least one surfactant selected from the group consisting of organomodifled siloxane non-ionic surfactants, polyalkoxylated sorbitan carboxyl ales, alkyl sulfate surfactants, alcohol ethoxyiate surfactants, polysorbate surfactants and combinations thereof.

17. The method of claim 1, wherein the composition is comprised of from about 0.01% to about 30% or from about 0.01% to about 10% by weight surfactant.

18-19. (canceled)

20. The method of claim 1, wherein the contacting is achieved by spraying the composition onto the beeswax, beehive and/or beehive equipment surface, immersing the beeswax, beehive and/or beehive equipment surface in the composition, or fogging the beeswax* beehive and/or beehive equipment surface with the composition.

21. The method of claim 1, wherein the composition is activated by subjecting the composition to cold plasma ionizing during a fogging process before the composition reaches the beeswax, beehive and/or beehive equipment surface.

22-23. (canceled)

24. The method of claim 1, wherein the composition is additionally comprised of at least one viscosifying agent and/or gelling agent.

25. The method of claim 24, wherein the composition is comprised of from about 0.01% to about 10.0% by weight or from about 0.1% to about 5.0% by weight in total of viscosifying agent and/or gelling agent.

26-49. (canceled)
